(19) United States
(12) Patent Application Publication
(10) Pub. No.: US 2017/0215747 A1
(43) Pub. Date: Aug. 3, 2017

(54) OPTICAL VITAL SIGNS SENSOR

(71) Applicant: KONINKLIJKE PHILIPS N.V., EINDHOVEN (NL)

(72) Inventors: Cornelus Hendricus Bertus Arnoldus van Dinther, Mierlo (NL); David Antoine Christian Marie Roovers, Eindhoven (NL)

(21) Appl. No.: 15/411,096

(22) Filed: Jan. 20, 2017

Related U.S. Application Data
(63) Continuation of application No. PCT/EP2017/050740, filed on Jan. 16, 2017.

(30) Foreign Application Priority Data
Feb. 1, 2016 (EP) 16153692.5

(51) Int. Cl. A61B 5/0295; A61B 5/00; A61B 5/1455; A61B 5/026; A61B 5/024

(52) U.S. Cl. CPC A61B 5/0295; A61B 5/0261; A61B 5/02427; A61B 5/14552; A61B 5/681; A61B 5/6815; A61B 5/6826

(57) ABSTRACT

An optical vital signs sensor embodied as a PPG sensor is provided. The optical vital signs sensor comprises a light source to generate light at at least two wavelengths, a photo detector unit configured to detect the light of the at least two wavelengths, a contact surface to be placed against a skin of the user and an off-skin detection unit configured to detect whether the contact surface is in contact with the skin of the user based on output signals of the photo detector at the at least two wavelengths.

### CROSS REFERENCE TO RELATED APPLICATION

The present application is a Continuation of International Application No. PCT/EP2017/050740, filed Jan. 16, 2017, which claims priority to European Application No. 16153692.5, filed Feb. 1, 2016. These applications are incorporated herein by reference, for all purposes.

### TECHNICAL FIELD

The embodiment relates to an optical vital signs sensor for monitoring vital signs of a user.

### BACKGROUND

Optical heart rate sensors are well known to monitor or detect vital signs like a heart rate of a user. Such a heart rate sensor can be based on a photoplethysmograph (PPG) sensor and can be used to acquire a volumetric organ measurement. By means of pulse oximeters, changes in light absorption of a human skin are detected and based on these measurements a heart rate or other vital signs of a user can be determined. The PPG sensors comprise a light source like a light emitting diode (LED) which is emitting light into the skin of a user. The emitted light is scattered in the skin and is at least partially absorbed by the blood. Part of the light exits the skin and can be captured by a photo detector. The amount of light that is captured by the photo detector can be an indication of the blood volume inside the skin of a user. A PPG sensor can thus monitor the perfusion of blood in the dermis and subcutaneous tissue of the skin through an absorption measurement at a specific wave length. If the blood volume is changed due to the pulsating heart, the scattered light coming back from the skin of the user is also changing. Therefore, by monitoring the detected light signal by means of the photo detector, a pulse of a user in his skin and thus the heart rate can be determined. Furthermore, compounds of the blood like oxygenated or de-oxygenated hemoglobin as well as oxygen saturation can be determined.

The PPG sensor can be implemented for example in a smart watch and can be placed in direct contact with the skin of the user. If the PPG sensor is, however, not anymore in direct contact with the skin of the user, e.g. if a loss of skin contact has occurred, the output of the photo detector can not be used to detect vital signs of a user.

US 2002/0137995 A1 discloses a system for detecting sensor off conditions in a PPG sensor. The system detects reflected light at two different wavelengths and determines a correlation coefficient to determine whether a sensor off condition is present.

WO 2009/088799 A1 discloses an optical vital signs sensor comprising a light source, photo detector as well as an off-skin detection unit which uses a comparison of DC components at a first wavelength with DC components at a second wavelength to determine whether or not the sensor is in contact with the skin of the user.

US 2004/0097797 A1 discloses an optical vital signs sensor having a light source and a photo detector.

EP 1 792 564 A1 discloses an optical vital signs sensor having a light source as well as a photo diode.

US 2014/0288390 A1 discloses an optical vital signs sensor having a light source as well as a photo diode.

US 2014/0176944 A1 discloses an optical vital signs sensor of a user which comprises a light source as well as a detector.

### SUMMARY

It is an object of the embodiment to provide an optical vital signs sensor which is able to reliably detect when the sensor is not in contact with a skin of a user.

According to an aspect of the embodiment an optical vital signs sensor is provided to measure or determine vital signs of a user. The optical vital signs sensor is a photoplethysmographic sensor (PPG). A light source is configured to generate light at least two wavelengths which is directed towards a skin of the user. The sensor also comprises a photo detector unit configured to detect light at the at least two wavelengths, said light is indicative of a reflection of light emitted in or from the skin of the user, wherein the reflected light is light at the at least two wavelengths. The optical vital signs sensor comprises a contact surface configured to be placed against a skin of a user. The light from the light source is directed towards the skin of the user via the contact surface. The optical vital signs sensor comprises an off-skin detection unit configured to detect whether the contact surface is in contact with the skin of the user based on output signals from the photo detector unit at the at least two wavelengths. The off-skin detection unit is configured to compare DC components of the output signal of the photo detector unit at a first wavelength with DC components of the output signal of the photo detector unit at a second wavelength in order to detect whether the contact surface is in contact with the skin of the user. The first wavelength corresponds to green light and the second wavelength corresponds to red light. The off-skin detection unit comprises a first comparing unit configured to compare the DC components of the output signal of the photo detector at a first wavelength with a DC component of the output signal of the photo detector at a second wavelength. The off-skin detection unit comprises a DC removal unit configured to remove the DC components of the output signals of the photo detector at the first and second wavelength. The off-skin detection unit comprises a buffer with a first and second portion each configured to store AC components of the output signal of the photo detector at the first and second wavelength. The off-skin detection unit furthermore comprises a root mean square determining unit configured to determine the root mean square of the AC components stored in the first buffer portion and the root mean square of the AC components stored in the second buffer portion. The off-skin detection unit comprises a second comparing unit configured to compare the root mean square of the AC component in the first buffer portion with the root mean square of the AC components stored in the second buffer portion to determine whether the contact surface is in contact with the skin of a user.

According to a further aspect of the embodiment, the light source comprises at least two light units each configured to emit light substantially at one wavelength.

According to a further aspect of the embodiment, the light source comprises a tunable light unit configured to emit light substantially at one wavelength.

According to a further aspect of the embodiment, the light source comprises a white band light unit configured to emit light at at least two wavelengths. The photo detector comprises a tunable filter configured to tune to the at least two wavelengths of the light unit.

According to a further aspect of the embodiment, the optical vital signs sensor comprises a motion detection unit configured to detect a motion level of a user.

According to a further aspect of the embodiment, a method of measuring or determining vital signs of a user with an optical vital signs sensor configured to measure or determine vital signs of a user is provided. The optical vital signs sensor is a photoplethysmographic sensor PPG and has a contact surface. Light at the at least two wavelengths is generated and is directed towards a skin of the user. Light at the at least two wavelengths is detected. The light is indicative of a reflection of light emitted in or from the skin of the user. The reflected light is light at the at least two wavelengths. An off-skin detection unit is provided to detect whether the sensor (its contact surface) is in contact with the skin of a user based on output signals at the two wavelengths.

According to an aspect of the embodiment, a computer program product comprising a computer readable memory storing computer program code means for causing the optical vital signs to carry out the steps of measuring or determining vital signs of a user as described above is provided.

According to an aspect of the embodiment, the vital signs sensor comprises a LED based PPG sensor. The LED light penetrates the skin of the user, is reflected and some of it can reach a photo detector. The output of the photo detector can be used to monitor a blood volume fraction and blood compounds like oxygenated and de-oxygenated hemoglobin. In particular, the amount of absorption or reflectance of the light from the LED light source can be used to determine the heart rate as well as the blood volume fraction or blood compounds. The heart rate relates to the blood volume fraction. Furthermore, the PPG sensor according to the embodiment is therefore an optical sensor allowing a non-invasive measurement of vital signs of a user.

If a PPG sensor which is designed to operate in direct contact with the skin of the user is not in direct contact with the skin of the user anymore, the output of the photo detectors will include artifacts such that a detection of vital signs of a user is not possible anymore. According to the embodiment, if an off-skin condition is detected, the output signals of the photo detector can be ignored. Furthermore, optionally, the PPG sensor can be put into a stand-by mode or the light units can be switched off in order to reduce the power consumption.

It shall be understood that a preferred embodiment of the present embodiment can also be a combination of the dependent claims or above embodiments or aspects with respective independent claims.

These and other aspects of the embodiment will be apparent from and elucidated with reference to the embodiment(s) described hereinafter.

### BRIEF DESCRIPTION OF THE DRAWINGS

In the following drawings:

FIG. 1 shows a basic representation of an operational principle of a vital signs sensor,

FIG. 2 shows a block diagram of a vital signs sensor according to an aspect of the embodiment,

FIG. 3 shows a flow chart of an off-skin detection in a vital signs sensor according to an aspect of the embodiment,

FIG. 4 shows a graph indicating a blood absorption as function of wavelength,

FIG. 5 shows a graph indicating DC levels for a first and second wavelength on different surfaces,

FIG. 6 shows a graph indicating DC levels at two different wavelengths on different colour surfaces,

FIG. 7 shows a flow chart of an off-skin detection in a vital signs sensor according to an aspect of the embodiment, and

FIG. 8 shows a block diagram of an off-skin detection unit in an optical vitals signs sensor according to an aspect of the embodiment

### DETAILED DESCRIPTION OF EMBODIMENTS

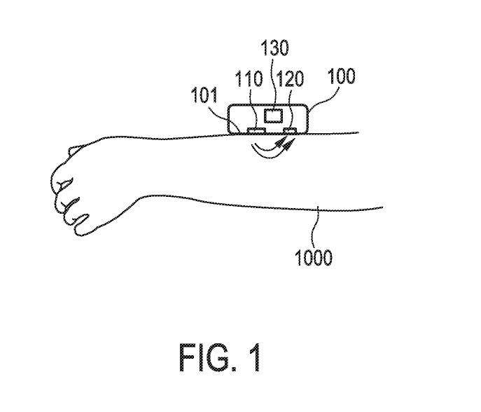

FIG. 1 shows a basic representation of an operational principle of an optical vital signs sensor. In FIG. 1, the optical vital signs sensor, e.g. a heart rate sensor 100, with its contact surface 101 is arranged or placed on for example an arm of a user. The contact surface 101 can be (directly) placed onto the skin 1000 of the user. The heart rate sensor 100 comprises at least one light source 110 and at least one photo detector 120. The light source 110 emits light via the contact surface 101 onto or in the skin 1000 of a user. Some of the light is reflected and the reflected light can be detected by the photo detector 120. Some light can be transmitted through tissue of the user and be detected by the photo detector 120. Based on the reflected light, vital signs of a user like a heart rate can be determined. An off-skin detection unit 130 analyzes the output of the at least one photo detector 120 to detect whether the sensor 100 is in contact with the skin 1000 of the user.

The output signal of the PPG sensor gives an indication on the blood movement in vessels of a user. The quality of the output signal of the PPG sensor can depend on the blood flow rate, skin morphology and skin temperature. In addition, optical losses in the PPG sensor may also have an influence on the quality of the output signal of the PPG sensor. The optical efficiency of the PPG sensor can depend on reflection losses when light penetrates from one media into another. Furthermore, scattering of light at the surface of the skin of the user may also have an influence on the optical efficiency of the PPG sensor.

The PPG sensor or optical vital signs sensor according to an aspect of the embodiment can be implemented as a device that requires a contact with the skin of the user such as a wrist device (like a watch or smart watch). The optical vital signs sensor can also be implemented as a device worn behind the ear of a user, e.g. like a hearing aid or a device clamped to a finger.

The PPG sensor can be a wavelength-diverse PPG sensor, which determines the vital signs of the user based on samples at different wavelengths. The PPG sensor can emit light at different wavelengths and detects the accordingly reflected light. The light source can comprise a plurality of light units like LEDs which can be activated to emit light.

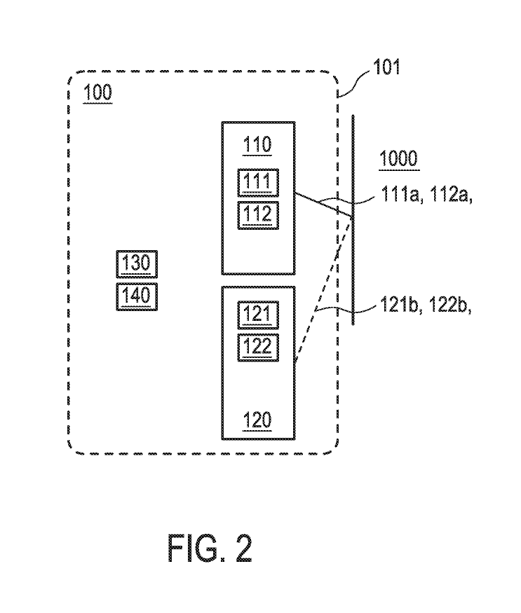

FIG. 2 shows a block diagram of an optical vital signs sensor according to an aspect of the embodiment. The optical vital signs sensor 100 may comprise a contact surface 101 which can be placed in (direct) contact with the skin 1000 of a user. The optical vital signs sensor comprises a light unit 110 which can have two light emitting diodes 111-112. These two light emitting diodes 111-112 may emit light 111a, 112a at two different wavelengths P1, P2. Alternatively, the light unit 110 may also comprise one tunable light emitting diode which can emit light 111a, 112a at two different wavelengths P1, P2.

The optical vital signs sensor 100 furthermore comprises a photo detector unit 120 which is able to detect the reflected light 121a-122b. The light unit 110 can be able to emit light 111a-112a at two wavelengths. The photo detector 120 may comprise two different photo diodes 121-122 which are able to detect the reflected light at the two different wavelengths P1, P2. The output of the photo detector 120 is forwarded to an off-skin detection unit 130 which serves to detect when the optical vital signs sensor 100 is not in direct contact with the skin 1000 of the user. The operation of the PPG sensor 100 can be controlled according to the output signal of the off-skin detection unit 130.

The PPG sensor 100 can also comprise a motion sensor 140 for detecting a motion of the PPG sensor. The output of the motion sensor 140 can be used to activate the PPG sensor after it has been deactivated after an off-skin detection. In other words, the output of the motion unit 140 (which can be implemented as an accelerator) can be used to activate the off-skin detection.

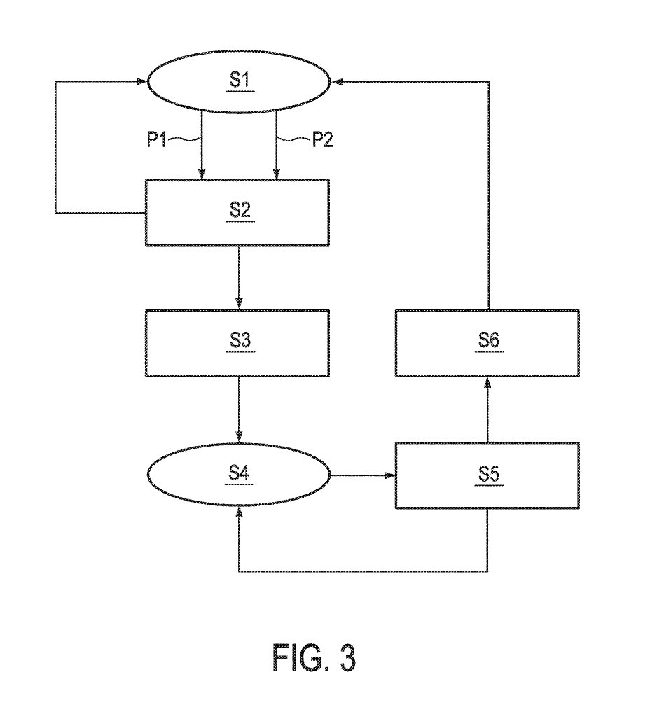

FIG. 3 shows a flow chart of an off-skin detection in a vital signs sensor according to an aspect of the embodiment. In step S1, the PPG sensor 100 is activated and light is emitted by the light units at a first wavelength P1 (525 nm, green) as well as at a second wavelength P2 (630 nm, red). In other words, the PPG sensor 100 is emitting light at 525 nm and 630 nm. Step S1 can be initiated when the optical vital signs sensor 100 is switched on or alternatively when a motion of the optical vital signs sensor is detected by an accelerometer 140 in the PPG sensor 100.

In step S2, an off-skin detection is performed by the off-skin detection unit 130 based on an output signal of the photo detector 120 at the first and second wavelength P1, P2. If no off-skin condition is detected, the flow continues to step S1. If, however, an off-skin detection is positive, the flow continues to step S3 where the PPG sensor 100 is switched off. Then the flow continues to step S4. In step S4, the PPG sensors 100 are off. Then the flow can continue to step S5 where the PPG sensors can be switched on again, for example if a movement has been detected. If the PPG sensors 100 are not switched on, then the flow continues to step S4. However, if the PPG sensors 100 are switched on again, the flow continues to step S6 as the PPG sensors are activated. Then the flow continues to step S1.

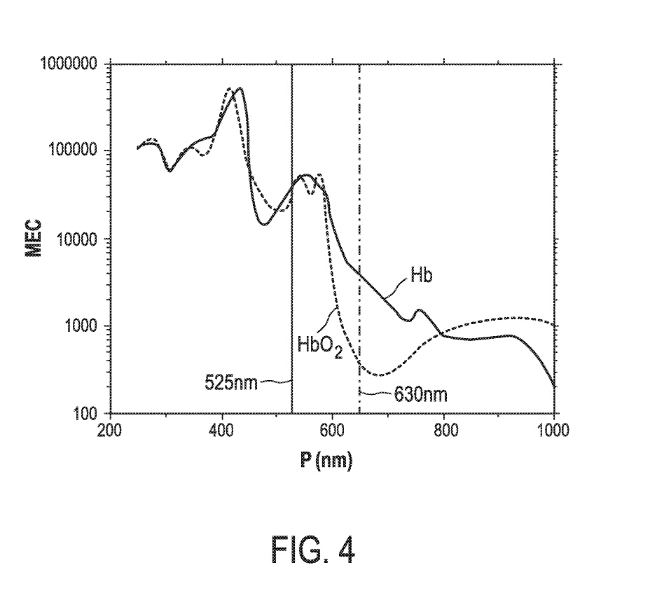

FIG. 4 shows a graph indicating a blood absorption as function of wavelength in an optical vital signs sensor according to an aspect of the embodiment in a tissue. In FIG. 4, the wavelength P (nm) as well as a molar extinction coefficient MEC is depicted. The molar extinction coefficient MEC serves as an indication of blood absorption. In FIG. 4, two wavelengths, namely 525 (green) and 630 nm (red) are depicted explicitly. In FIG. 4, the oxinated HbO₂ and the deoxinated Hb curves are depicted.

According to the embodiment, the off-skin detection can be performed based on a comparison of the DC levels of the output signals of the photo detector 120 at the first and second wavelength P1 (525 nm), P2 (630 nm). Optionally, the DC levels of the output signals of the photo detector can be normalized by the power of the light units as well as an ADC gain.

As can be seen from FIG. 4, a reflected light with the colour red (630 nm) is less absorbent than light of the colour green (525 nm). In other words, red light is more reflected and has a larger DC component than green light.

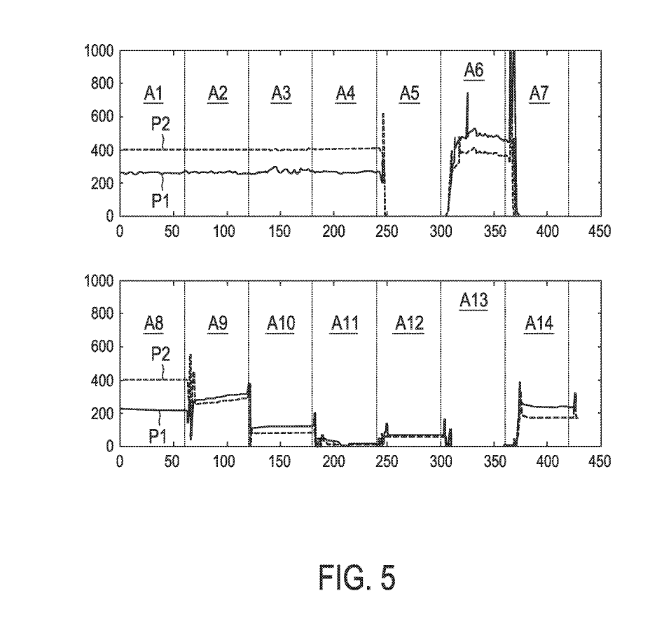

FIG. 5 shows a graph indicating DC levels of an output signal of the photo detector of an optical vital signs sensor according to an aspect of the embodiment for different surfaces. In FIG. 5, 14 different surfaces A1-A14 are depicted. The first surface A1 is the wrist of a human in rest. The second surface A2 is the wrist of a human in motion. The third surface A3 is also a wrist in motion. The fourth surface A4 is a wrist at rest. The fifth surface A5 is present when the PPG sensor is facing upwards, i.e. there is no surface present. The sixth surface A6 is a table. The seventh surface A7 was detected when the PPG sensor is facing sidewards. The eighth surface A8 is a wrist at rest. The ninth surface A9 is a table. The tenth surface A10 is black plastic. The eleventh surface A11 is a grey carpet. The twelfth surface A12 is brown leather. The thirteenth surface A13 is grey cotton. The fourteenth surface A14 is white paper.

As can be seen from FIG. 5, the DC component at the second wavelength P2 is only higher than the DC component of the first wavelength P1 when the PPG sensor 100 is placed at a wrist of a user. In other words, if the PPG sensor 100 is not placed against a wrist of a user, the DC component at the first wavelength P1 is higher than the DC component at the second wavelength P2.

Accordingly, a comparison of the DC components at the first and second wavelength P1, P2 can be used in the off set detection unit 13 to detect whether the PPG sensor is placed on a wrist or skin of a user.

The off-skin detection unit 130 compares the DC component of the output signals of the photo detector 120 at the first and second wavelength P1, P2. If the DC component of the second wavelength (630 nm, red) is larger than the DC component of the first wavelength (525 nm, green), then the PPG sensor is in contact with the skin. If, however, the DC component at the first wavelength P1 (525 nm, green) is larger than the DC component at the second wavelength P2 (630 nm, red), then the PPG sensor 100 is not in contact with the skin and is therefore off-skin. Moreover, if the DC components of the first and second wavelength P1, P2 are both below a threshold value (close to zero), the sensor 100 is also not in contact with the skin.

It can be seen from FIG. 5 that the DC component at the second wavelength P2 is higher than the DC component at the first wavelength P1 when the sensor is at rest or in motion as long as it is placed against the wrist of a user. At the fifth, seventh and thirteenth surface it can be seen that both DC components at the first and second wavelength P1, P2 are close to zero. In other words, the DC components at the first and second wavelength P1, P2 are close to zero if the PPG sensor is facing less reflective surfaces or not facing a surface at all.

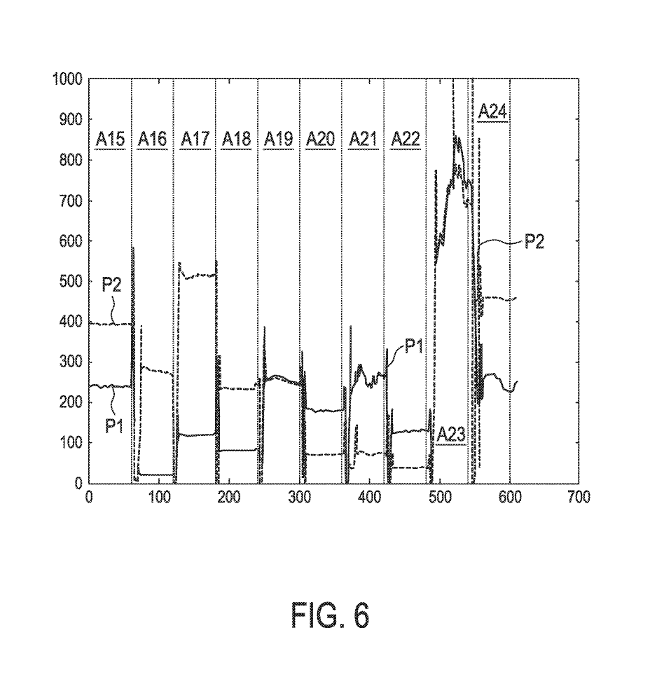

FIG. 6 shows a graph indicating DC levels at two different wavelengths for an optical vital sensor according to an aspect of the embodiment for different surfaces. In FIG. 6, the DC levels of the first and second wavelength P1 (525 nm, green) and P2 (630 nm, red) are depicted for different coloured paper surfaces. The fifteenth surface A15 is the left wrist at rest. The sixteenth surface A16 is a red surface. The seventeenth surface A17 is a lighter red surface. The eighteenth surface A18 is an orange surface. The nineteenth surface A19 is a yellow surface. The twentieth surface A20 is a green surface. The twentyfirst surface A21 is a light blue surface. The twenty-second surface A22 is a blue surface. The twenty-third surface A23 is a pink surface and the twenty-fourth surface is the right wrist at rest. According to the aspect of the embodiment based on FIG. 5, an off-skin is detected if a DC component at the second wavelength P2 (red) is higher than the DC component at the first wavelength (green). Therefore, it must be ensured that the off-skin detection unit 130 can also take into account colours of other different surfaces. As can be seen in FIG. 6, care must be taken for red surfaces, light red surfaces and orange surfaces. The red and orange surfaces may lead to false negatives, i.e. the DC of red is larger than the DC of green (thus on-skin is detected), while the device is off-skin facing a colored surface.

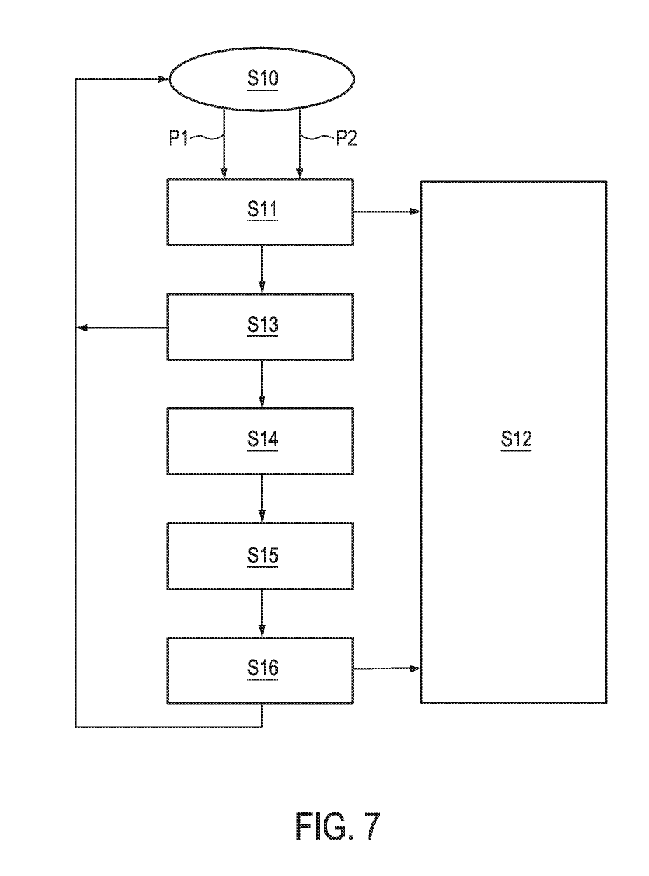

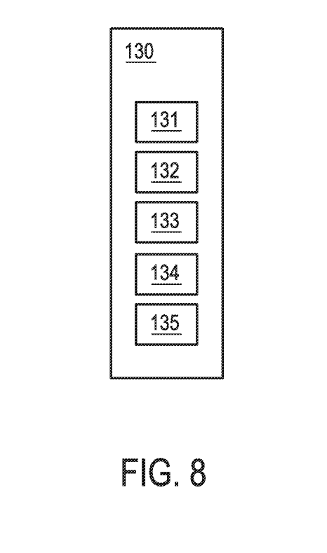

FIG. 7 shows a flow chart of an off-skin detection in a vital signs sensor according to an aspect of the embodiment. In step S10, the PPG sensors are on and the light units of the PPG sensor emit light at a first and second wavelength P1 (525 nm) and P2 (630 nm). In step S11, the DC component at the second wavelength P2 are multiplied by a constant factor C1 and are then compared to the DC component at the first wavelength P1. If the DC component at the first wavelength P1 is larger than the DC component at the second wavelength P2 multiplied by the factor C₁, then the PPG sensors are switched off (step S12). If this is not the case, then the flow continues to step S13, where a motion level is determined and compared to a threshold C2. If the motion level is below the threshold C2, then the flow continues to step S14, otherwise the flow continues to step S10. In step S14, the DC component at the first and second wavelength P1, P2 is removed. This can for example be performed by a high-pass filtering. Then the flow continues to step S15, where the remaining AC components at the first and second wavelength P1, P2 are stored in two circular buffers 133, 134. Then the flow continues to step S16. Here, the root mean square S is computed based on the data in the first and second buffer 133, 134. The results thereof are compared. If the root mean square RMS of the first buffer 133 is smaller than the root mean square RMS of the second buffer 134 times a constant C3, the flow continues to step S12 where the PPG sensors are switched off. Otherwise, the flow continues to step S10. FIG. 8 shows a block diagram of an off-skin detection unit in an optical vitals signs sensor according to an aspect of the embodiment. The off-skin detection unit 130 comprises a first comparing unit 131 which serves to compare the DC component of the output signal of the photo detector 120 at the first wavelength P1 with the DC component of the output signal of the photo detector 120 at the second wavelength P2 multiplied by a constant factor C1. The off-skin detection unit 130 furthermore comprises a DC removal unit 132 for removing the DC component of the output signals of the photo detector 120 at the first and second wavelength P1, P2 such that only the AC components remain. The AC components of the output signal of the photo detector 120 at the first wavelength P1 are stored in a buffer 133 (in particular in a first portion of the buffer) and the AC components of the output signal of the photo detector 120 are stored in the buffer 133 (in particular in a second portion of the buffer). A root mean square unit 134 calculates the root mean square RMS of the AC components in the buffer 133 (in particular in the first and second portion of the buffer) and a second comparing unit 135 compares the results of the AC components in the first buffer portion with the root mean square of the AC components in the second buffer portion multiplied by a constant C3. If the root mean square of the AC components in the first buffer portion is smaller than the root mean square of the AC components in the second buffer 134 times the constant C3, then the PPG sensor 100 can be switched off.

It should be noted that light at the first wavelength (green, 525 nm) is more absorbed by blood than light at the second wavelength (red, 630 nm). Accordingly, variations in the blood volume can be more visible at the first wavelength than at the second wavelength P1, P2. These variations can be determined by calculating the root mean square of the signal. A larger root mean square value will indicate a higher variation. It should be noted that any other surfaces than human tissue will not absorb these characteristics as there is no variation in a blood volume. In fact, a variation of any other surfaces will be proportionate to the reflected light as measured in the DC signal. According to an aspect of the embodiment, the DC components as well as the AC components at the first and second wavelength P1, P2 be used during an off-skin detection. Accordingly, a reliable off-skin detection is provided according to an aspect of the embodiment.

According to an aspect of the embodiment, the usage of the motion level in the flow chart according to FIG. 7 enables a more robust and reliable off-skin detection as it ensures that the root mean squares of the signals are only computed if no motion is detected. The reason is that during motion, the root mean square of the AC component at the first wavelength P1 can be higher than that at the second wavelength P2.

The comparison of the DC signals can be performed on a sample basis. However, it is also possible to measure the DC components over a certain period of time. Accordingly, a smooth version, an average version or other mappings of the DC signals are also possible.

Furthermore, the PPG sensor may be switched on and off after measuring the signals for a period of time or after other constraints. It should be noted that it is also possible to use more than two wavelengths.

Other variations of the disclosed embodiment can be understood and effected by those skilled in the art in practicing the claimed embodiment from a study of the drawings, the disclosure and the appended claims.

In the claims, the word “comprising” does not exclude other elements or steps and in the indefinite article “a” or “an” does not exclude a plurality.

A single unit or device may fulfil the functions of several items recited in the claims. The mere fact that certain measures are recited in mutual different dependent claims does not indicate that a combination of these measurements cannot be used to advantage. A computer program may be stored/distributed on a suitable medium such as an optical storage medium or a solid state medium, supplied together with or as a part of other hardware, but may also be distributed in other forms such as via the internet or other wired or wireless telecommunication systems.

Any reference signs in the claims should not be construed as limiting the scope.

1. Optical vital signs sensor (100) configured to measure or determine vital signs of a user, said optical vital signs sensor being a photoplethysmographic sensor (PPG), comprising:
a light source (110) configured to generate light at at least two wavelengths (P1, P2) which is directed towards a skin (1000) of the user (1000),
a photo detector unit (120) configured to detect light at the at least two wavelengths, wherein said light being indicative of a reflection of light emitted in or from the skin (1000) of the user, wherein the reflected light is light at at least two wavelengths (P1, P2), a contact surface (101) configured to be placed against a skin (1000) of a user, wherein the light from the light source (110) is directed towards the skin (1000) of the user via the contact surface (101), and an off-skin detection unit (130) configured to detect whether the contact surface (101) is in contact with the skin (1000) of the user based on output signals from the photo detector unit (102) at the at least two wavelengths (P1, P2), wherein the off-skin detection unit (130) is configured to compare DC components of the output signal of the photo detector unit (120) at a first wavelength (P1) with DC components of the output signal of the photo detector unit (120) at a second wavelength (P2) to detect whether the contact surface (101) is in contact with the skin (1000) of a user,
wherein the off-skin detection unit (130) comprises a first comparing unit (131) configured to compare the DC components of the output signal of the photo detector (120) at a first wavelength (P1) with a DC component of the output signal of the photo detector unit (120) at a second wavelength (P2), a DC removal unit (132) configured to remove the DC components of the output signals of the photo detector unit (120) at the first and second wavelengths (P1, P2), a buffer (133) having a first and second buffer portion each configured to store AC components of the output signal of the photo detector unit (120) at the first wavelength (P1) and at the second wavelength (P2), a root mean square determining unit (134) configured to determine the root mean square of the AC components stored in the first buffer portion and the root mean square of the AC components stored in the second buffer portion, and a second comparing unit (135) configured to compare the root mean square of the AC components in the first buffer (133) and the root mean square of the AC components stored in the second buffer (133) to determine whether the contact surface (101) is in contact with the skin (1000) of the user,
wherein the first wavelength (P1) corresponds to green light and the second wavelength (P2) corresponds to red light.

2. Optical vital signs sensor (100) according to claim 1, wherein the light source (110) comprises at least two light units (111-11i) each configured to emit light (211-21i) substantially at one wavelength (P1, P2).

3. Optical vital signs sensor (100) according to claim 1, wherein the light source (110) comprises a tuneable light unit configured to emit light substantially at one wavelength.

4. Optical vital signs sensor (100) according to claim 1, wherein the light source (110) comprises a wideband light unit configured to emit light at at least two wavelengths, and wherein the photo detector unit (120) comprises a tuneable filter (125) configured to tune to the at least two wavelengths of the light unit (110).

5. Optical vital signs sensor according to claim 1, further comprising a motion detection unit (140) configured to detect a motion level of a user, wherein the optical vital signs sensor can be activated based on the output signal of the motion detection unit (140).

6. Method of measuring or determining vital signs of a user with an optical vital signs sensor (100) configured to measure or determine vital signs of a user, said optical vital signs sensor being a photoplethysmographic sensor (PPG) and has a contact surface (101) configured to be placed against a skin (1000) of a user, comprising the steps of:
generating light at a plurality of wavelengths which is directed towards a skin (1000) of the user (1000),
detecting light at a plurality of wavelengths, wherein said light being indicative of a reflection of light emitted in or from the skin (1000) of the user, wherein the reflected light is light at a plurality of wavelengths, and
detecting whether the contact surface (101) is in contact with the skin (1000) of the user based on output signals from the photo detector unit (102) at the at least two wavelengths (P1, P2) by comparing DC components of the output signal of the photo detector unit (120) at a first wavelength (P1) with DC components of the output signal of the photo detector unit (120) at a second wavelength (P2) and by comparison AC components of the output signal of the photo detector unit (120) at a first wavelength (P1) with AC components at a second wavelength (P2) to detect whether the contact surface (101) is in contact with the skin (1000) of a user,
wherein the first wavelength (P1) corresponds to green light and the second wavelength (P2) corresponds to red light.

7. A computer program product comprising a computer readable memory storing computer program code means for causing the optical vital signs sensor according to claim 1 to carry out the steps of measuring or determining vital signs of a user according to claim 6.
